(19) United States
(12) Patent Application Publication
(10) Pub. No.: US 2022/0082505 A1
(43) Pub. Date: Mar. 17, 2022

(54) Rapid Methanol Detection Device

(71) Applicants: Joseph James Whitlock, Manlius, NY (US); Richard Hammitt, Spring, TX (US)

(72) Inventors: Joseph James Whitlock, Manlius, NY (US); Richard Hammitt, Spring, TX (US)

(21) Appl. No.: 17/024,620

(22) Filed: Sep. 17, 2020

(51) Int. Cl. G01N 21/78; C12Q 1/26; C12Q 1/32

(52) U.S. Cl. CPC G01N 21/78; C12Q 1/32; C12Q 1/26

(57) ABSTRACT

A test strip that provides rapid detection device of a target substance includes a preconditioned, chemically impregnated membrane, that when placed in contact with suspect liquid, undergoes a chemical reaction that results in a visual indication of the presence of target substance. In an embodiment, the formulation to be applied to the membrane includes: alcohol oxidase of 0.001 to 1.00 wt. %, formaldehyde dehydrogenase of 0.01 to 1.00 wt. %, nicotinamide adenine dinucleotide of 0.001 to 1.00 wt. %, diaphorase or phenazine methosulfate of 0.00001% to 1.00 wt. %, and tetranitro blue of 0.001 to 1.00 wt. %.

### CROSS-REFERENCE TO RELATED APPLICATION

This application claims priority to U.S. Provisional Patent Application Ser. No. 62/897,351, filed on Sep. 8, 2019 and entitled “Methanol Detection Device,” the entire disclosure of which is incorporated herein by reference.

### GOVERNMENT FUNDING

Not Applicable

### FIELD OF INVENTION

The present disclosure is directed generally to a rapid methanol detection test strip and method.

### BACKGROUND OF INVENTION

Methanol is a toxic chemical that is sometimes included as a low-cost diluent in products containing ethanol or contaminates ethanol when weak processes are used in ethanol distillation. The median lethal dose of methanol is 1-2 ml/kg body weight of pure methanol, or about 3 ounces (100 ml) for your person. It's metabolized in the liver to formic acid, which destroys the optic nerve and attacks other parts of the central nervous system.

Today's consumer must worry about methanol contaminated products; such as hand sanitizer, alcoholic beverages, mouthwash, perfume, and household cleaners, to name a few. Legitimate businesses that produce these products employ expensive, lengthy, complicated tests to control product quality; while others forgo these practices in order to cut costs. Unfortunately, emergency room physicians across the world do not possess the appropriate point of care tests needed to rapidly diagnose and administer aid to patients exhibiting methanol toxidrome symptoms.

The Covid-19 pandemic has caused global social and economic disruptions, and widespread supply shortages aggravated by panic buying. Unfortunately, the supply and demand issues have caused some manufacturers of hand sanitizers to increase production and either overlook or deliberately fortify products with methanol where ethanol should be used. As of Aug. 28, 2020, the FDA has identified 171 hand sanitizers that are suspected to contain methanol. The problem is that the FDA is identifying these contaminated products after they have flooded the market and are in the hands of consumers. In addition, other regions of the world do not have the same regulations imposed by the FDA, and it's the people residing in these locations that are the most at risk.

The FDA warns that, “Consumers who have been exposed to hand sanitizer containing methanol and are experiencing symptoms should seek immediate treatment for potential reversal of toxic effects of methanol poisoning. Substantial methanol exposure can result in nausea, vomiting, headache, blurred vision, permanent blindness, seizures, coma, permanent damage to the nervous system or death.” There have been multiple reported incidents caused by toxic hand sanitizers that have appeared on the market since July. Additionally, other alcohol-containing products (i.e. household cleaners, mouthwash, perfumes, wiper fluid), contributed to the nearly 2.1 million human poison exposures in 2018 according to the US poison control hotline. Currently, there is no available bedside test to rapidly assess the presence of these toxic alcohols in the serum of patients and determine whether ingestion has occurred. Definitive laboratory quantification, if available at all, can take hours despite the need to start anti-methanol poisoning treatments immediately.

Another widely known product where methanol substitution can be hazardous to consumers is in alcohol sold for consumption. Alcohol has historically and continues to hold an important role in social engagement and bonding. In the United States alone, alcoholic beverage industry sales reached $253.8 billion. The total volume of alcohol consumed globally per year has increased by 70%, from 20,999 million liters in 1990, to 35,676 million liters in 2017. Included in these statistics is the growing rate of alcohol consumption in India, which increased by 38% between 2010 and 2017. The World Health Organization (WHO) has issued multiple travel advisories in the past two years warning travelers to foreign destinations of methanol tainted alcohol. Despite these warnings, there have been multiple news articles reporting the deaths of 19 people in Costa Rica between June and July of 2019, and at least 13 U.S. tourists in the Dominican Republic. This impacts tourism, and hospitality industries in regions where methanol-laced alcohol is a growing concern.

Traditional methanol detection methods require expensive apparatuses that make them inapplicable to common laboratory settings or to the general public (i.e. high-performance liquid chromatography, Fourier transform infrared spectrometry, and Gas Chromatography). In addition, the existing chemical methods for detecting methanol, such as the phosphorus chloride method, contain hazardous chemical reagents that are not suitable for the typical consumer to handle. Enzymes, on the other hand, are ideal as they are highly specific to the target; in this case methanol. Furthermore, once it is in aldehyde form (formaldehyde) the enzyme is highly specific in converting the formaldehyde and no other aldehyde to the color signal.

Several other methods to detect methanol exist, but each has its own shortcomings when compared to the proposed invention. The Tollens test for detecting formaldehyde uses silver nitrate and base by converting the silver into a mirror on glass. This yields attribute data (i.e. yes/no) and could not easily be converted to variable data (i.e. how much). One method for detecting ethanol in urine or blood is the ethyl glucuronide test, which detects what the body converts ethanol into. It requires a human body to convert the ethanol to a detectable method, although it could be done with a liver enzyme (UDP glucuronosyl transferase). A final pre-existing method is the phosphorous chloride method. Hydrogen chloride produced indicates the presence of an alcohol. Subsequent addition of potassium dichromate and sulfuric acid can distinguish between a primary/secondary alcohol and tertiary alcohol by color, but this method is nonspecific for methanol and hazardous.

To date, there are several methanol assay kits available on the market today; however, these kits are geared mostly towards research institutes and are not embodied in a rapid device such as a test strip. For example, the ABCAM methanol assay kit retails for $590 for 100 tests, must be stored at −20 C, and involves a complex multistep process upon which one must wait 30 minutes for a result. One device that is geared towards the public is sold by Mile High Distilling for $85 per test. This test kit contains an empty bottle that must be filled with the spirit sample by the brewer, and subsequently mailed back to the laboratory for testing that takes two to three weeks for results. Lastly, “Alert” by Neogen, is a screening tool for the rapid detection of potentially harmful methanol contamination in spirits, beers and wine and retails for $413.50 for 20 tests. Customers must follow a series of detailed steps in order to arrive at a result in approximately 10 minutes.

In regard to detecting substances in alcoholic beverages, there have been numerous patents filed to address the widespread use of “date rape” drugs that are plaguing global society. In these publications the suggested devices are geared towards detecting the presence of chemicals commonly found in “date rape” drugs such as GBH, GMB, etc. Two such patents claim to be able to detect the presence of these compounds via an electrical probe or optical detection; U.S. Pat. No. 2011/0054801 A1 by Hilborne and U.S. Pat. No. 9,228,991 B2 by Patolsky, respectively. Both of the devices taught in these references rely on pre-recorded tables of experimentally determined values under carefully controlled experimental settings that are not accurately transferable to real world applications.

The additional prior art listed below relates generally to the present invention:

U.S. Pat. No. 8,241,575 B2 by Murray, claims a molecularly imprinted polymer sensor device for measuring and detecting a wide variety of target molecules in a fluid.

U.S. Pub. No. 2012/0070901 A1 by Bradley, claims a drug detection device in the form factor of a straw or stir stick that is capable of changing colors in the presence of a date rape drugged beverage.

U.S. Pub. No. 2012/0164278 A1 by Abramson, claims a beverage container configured with various sections to test for a contaminated beverage.

WO 2017/132614 filed by undercover colors, makes broad claims about an apparatus for detecting many targeted substances; however, methanol is never listed.

An abandoned patent application, 20090120808, from Wu, claims to detect the methanol concentration of a solution by measuring the current value resulting after a two-stage reaction. Wu relies on taking the difference of initial and final current measurements and subtracting the ethanol noise based on pre-recorded tables to determine the presence of methanol in a consumer beverage. Wu does not disclose at least a test strip or a dye that changes color upon being reduced to detect the presence of ethanol in a beverage.

All of the prior art summarized above is focused on detecting chemicals contained in “date rape” drugs such as GBH, etc. Furthermore, given the sociological implication of the situation, the prior art is focused on providing solutions for obstacles that do not impact the situation that this device serves. For example, the prior art concerns itself with the need to test a beverage at multiple durations in order to ensure contamination has not occurred since the previous detection instance. Therefore expensive, continuously monitoring devices are not required and impractical for low cost applications. Additionally, the majority of the prior art is not capable of detecting the presence of methanol in an alcoholic beverage. Moreover, the large diversity of alcoholic and non-alcoholic beverages, and their mixtures, makes the prior art detection methods for chemical drugs spiked into these liquid samples extremely challenging.

The below journals, and articles are also considered relative to the claimed invention as they relate specifically to existing technologies utilized to detect methanol:

In “An Alcohol Oxidase Dipstick Rapidly Detects Methanol in the Serum of Mice,” by Hack, Early, and Brewer, the paper describes a dipstick that changes color in response to both methanol and ethanol. While the dipstick will change color at lower levels of methanol (7 mg/mL lower limit of full color change) than ethanol (22 mg/mL lower limit of full color change), the color change does not tell the observer whether it is ethanol or methanol giving the color change.

In “Critical Study of Methods for the Detection of Methyl Alcohol,” by Alexander Gettler, Gettler classifies tests for methyl alcohol into two groups: [A] that group in which the methyl alcohol must first be oxidized into formaldehyde before the tests are applied; [B] that group in which no oxidation is necessary. One major weakness in the study was Gettler had to preconcentrate the methanol before the test, something the Rapid Methanol Detection Device does not require.

In the paper by Gettler, all tests in group [A] involve exposure to metals or extreme pH, something that would not be viable for a rapid test like the one being proposed. Additionally, all tests in group [A] produce a small amount of acid in addition to the desired aldehyde, and the acid could give a false positive. The subsequent phenylhydrazines do give color, but only by using metals and pH extremes. The phenol are some alternate options for the color change, but some involve extreme pH. The alkaloid, aside from being controlled substances, are all using extreme pH. All of the remaining methods use metals, extreme pH, or both. Most of the methods in group [B] do not produce color. Some are specific to methanol, but the color tests require extreme pH and heat.

In “The Qualitative Detection of Methanol,” by Gakenheimer and Hartung, the rely on using metals for the color change. This could be used, but trace amounts of the permanganate going into solution would be an issue.

In “Approach to the Treatment of Methanol Intoxication”, by Kraut, a formate detector is mentioned, which is an alternative to the 3rd step, but did not describe the process.

In “A Color Test for Methanol,” by Giles, Hirst, Hoffmann, and Kapur, a simple color test for the analysis of methanol in serum and poisonous solutions using a three-enzyme system is described. However, the reagents were stored as a crushed, dry, cold mixture (stable at −4 C for greater than 3 months) until ready to dissolve.

Lastly, an abandoned Chinese patent application, CN2331971Y, “Quick detection test paper for methanol content in spirit,” discloses a test paper comprising a lamellar structure composed of a molecular sieve, a discoloration test paper encountering methanol, and a transparent plastic adhesive tape. The molecular sieve can only make methanol molecules in spirits pass through and contact the discoloration test paper to generate discoloration reaction; however, the chemistry behind said device is never mentioned.

Accordingly, there is a need in the art for a detection device that could alert an unsuspecting consumer to the presence of methanol in consumer products.

### BRIEF SUMMARY OF THE INVENTION

The present disclosure is directed to a test strip/membrane upon which a formulation is deposited that will give a color change on exposure to methanol.

The present invention comprises a novel mixture of enzymes and related materials which can selectively oxidize methanol, including in the presence of other alcohols, which results in the reduction of a dye and accompanying dye color change. The device can be used to detect methanol contamination in alcohol containing products, whether the presence of the methanol is due to a defect in the manufacturing process or whether it is the result of product tampering.

There are three steps to the reaction that leads to the color change:

Oxidation of Methanol

CH₃OH+O₂->CH₂O+H₂O₂

Oxidation of Formaldehyde/Reduction of NAD

2CH₂O+NAD⁺+H⁺->2CHO₂H+NADH

Detection of NADH

NADH+TB²⁺->NAD⁺+TB+H⁺

In the first step, methanol, as well as other primary alcohols in solution are oxidized selectively to the corresponding aldehyde. In the second step, formaldehyde, the product of the previous step's oxidation of methanol, is selectively oxidized to formic acid, while nicotinamide adenine dinucleotide is reduced. The reduced nicotinamide adenine dinucleotide is then oxidized while the dye is reduced. The dye changes color upon reduction, changing from yellow to purple when tetranitro blue is used as the dye.

According to an aspect is a test strip for colorimetrically indicating the presence of methanol in a solution it is in contact with, the test strip comprising: a reagent to oxidize methanol to formaldehyde, a reagent to oxidize formaldehyde to formic acid, a reagent to be reduced in the formaldehyde oxidation, a reagent to catalyze the reduction of a dye, and a dye that changes color when reduced.

According to an embodiment, the reagent used to oxidize methanol to formaldehyde is present in an amount of 0.001% to 1.00%.

According to an embodiment, the reagent used to oxidize methanol to formaldehyde is selected from one of the following: alcohol oxidase from Pichia pastoris, alcohol oxidase from Candida methanosorbosa, alcohol dehydrogenase from yeast, (2,2,6,6-tetramethylpiperidin-1-yl) oxyl (TEMPO), 2-azaadamantane-N-oxyl (AZADO), or 9-azabicyclo[3.3.1]nonane N-oxyl (ABNO).

According to an embodiment, the reagent used to oxidize formaldehyde to formic acid is present in an amount of 0.01 to 1.00%.

According to an embodiment, the reagent used to oxidize formaldehyde to formic acid is selected from: formaldehyde dehydrogenase from Pseudomonas putida, Methylococcus capsulatus, Pichia pastoris, Bacillus methanolicus, or Saccharomyces.

According to an embodiment, the reagent to be reduced in the formaldehyde oxidation is present in the amount of 0.001% to 1.00%.

According to an embodiment, the reagent to be reduced in the formaldehyde oxidation is nicotine adenine dinucleotide.

According to an embodiment, the reagent to catalyze the reduction of a dye is present in an amount of 0.00001% to 1.00%.

According to an embodiment, the reagent to catalyze the reduction of a dye is selected from: diaphorase from Clostridium kluyveri, Bacillus stearothermophilus, Bacillis megaterium, Escheria coli, Sus, Homo sapiens, or Rattus, or phenazine methosulfate.

According to an embodiment, the dye that changes color when reduced is present in an amount of 0.001% to 1.00%.

According to an embodiment, the dye that changes color when reduced is selected from: nitro blue, tetranitro blue, methylene blue, or 2,6-dichlorophenolindophenol.

According to an embodiment, the colorimetric indication occurs in less than or equal to 120 seconds when the presence of methanol is detected.

According to an embodiment, the test strip operates at an operating temperature range of 2 C to 80 C.

According to an embodiment, the test strip can be subjected to a storage temperature range of −20 C to 70 C.

According to an embodiment, the test strip can withstand mechanical vibration of 50 Hz for 30 minutes.

According to an embodiment, the test strip can withstand mechanical shock of 1 g.

According to an embodiment, the test strip shall withstand thermal shock of −20 C to 80 C.

According to an embodiment, the test strip shall withstand handling shock of a 20 lb drop test from a height of 1 meter.

These and other aspects of the invention will be apparent from the embodiments described below.

### BRIEF DESCRIPTION OF DRAWINGS

The present invention will be more fully understood and appreciated by reading the following Detailed Description in conjunction with the accompanying drawings, in which:

FIG. 1 is a plan view of a test strip, in accordance with an embodiment.

### DETAILED DESCRIPTION OF THE INVENTION

The present disclosure describes a rapid methanol detection device. All ratios, proportions, and percentages of components are expressed herein as w/w. In its preferred embodiment, the device detects the presence of methanol, and will not detect the presence of ethanol.

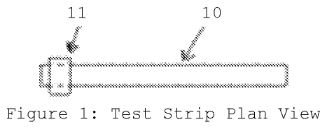

Referring to FIG. 1, in one embodiment, is a test strip 10 (it being understood that other form factors could also be used), having a preconditioned, chemically impregnated membrane 11 that when placed in contact with a suspect liquid undergoes a chemical reaction that results in a visual indication of the presence of target substance. In an embodiment, the formulation to be applied to the membrane comprises: alcohol oxidase of 0.001 to 1.000 wt. %, formaldehyde dehydrogenase of 0.01 to 1.00 wt. %, nicotinamide adenine dinucleotide of 0.001 to 1.00 wt. %, diaphorase or phenazine methosulfate of 0.00001% to 1.00 wt. %, and tetranitro blue of 0.001 to 1.00 wt. %.

A reagent used to oxidize methanol to formaldehyde that may include alcohol oxidase from Pichia pastoris, alcohol oxidase from Candida methanosorbosa, alcohol dehydrogenase from yeast, (2,2,6,6-tetramethylpiperidin-1-yl) oxyl (TEMPO), 2-azaadamantane-N-oxyl (AZADO), or 9-azabicyclo[3.3.1]nonane N-oxyl (ABNO). These and any other alcohol oxidation materials are referred herein as “methanol to formaldehyde catalyst”. The present invention also includes a reagent used to oxidize formaldehyde to formic acid that may include formaldehyde dehydrogenase from Pseudomonas putida, Methylococcus capsulatus, Pichia pastoris, Bacillus methanolicus, or Saccharomyces. These and any other aldehyde oxidation materials are referred herein as “formaldehyde to formic acid catalyst”. The present invention also includes a reagent to be reduced by the formaldehyde to formic acid catalyst and oxidized by the dye reduction catalyst and includes nicotinamide adenine dinucleotide. This and any other related ingredient are referred herein as “electron transport compound”. The present invention also includes a reagent to catalyze the reduction of a dye that may include diaphorase from Clostridium kluyveri, Bacillus stearothermophilus, Bacillis megaterium, Escheria coli, Sus, Homo sapiens, or Rattus, or phenazine methosulfate. These and any other dye reduction materials are referred herein as “dye reduction catalyst”. The present invention also includes a dye which changes color when reduced that may include nitro blue, tetranitro blue, methylene blue, or 2,6-dichlorophenolindophenol. These and any other dye are referred to as “reducible dye”. The formulation may be bound to a test strip, other form factor, or solvent borne.

Any numerical range recited herein is intended to include all sub-ranges subsumed therein. Plural encompasses singular, and vice versa.

The presence of alcohol oxidase from Pichia pastoris, formaldehyde dehydrogenase from Pseudomonas putida, phenazine methosulfate, nicotinamide adenine dinucleotide, and tetranitro blue gives a purple color in the presence of methanol, with the intensity of the color depending on the methanol concentration. This color change is highly specific for methanol and is useful in detecting the presence of methanol in the presence of ethanol and other structurally similar ingredients.

The percentage of the methanol to formaldehyde catalyst may vary from 0.001% to 1.00% by weight. The percentage of the formaldehyde to formic acid catalyst may vary from 0.01% to 1.00%. The percentage of the electron transport compound may vary from 0.001% to 1.00%. The percentage of the dye reduction catalyst may vary from 0.00001% to 1.00%. The percentage of the reducible dye may vary from 0.001% to 1.00%. At these levels, the catalyst provides a color that indicates the presence of methanol at 2.8% and above within 120 seconds. Excessive reducible dye may increase the background noise for the analysis.

Method of Making and Using:

In one embodiment, the test strip 10 is produced by dissolving the methanol to formaldehyde catalyst, the formaldehyde to formic acid catalyst, the dye reduction catalyst, the reducible dye, and the electron transport compound in a solution of bovine serum albumin and potassium phosphate, application of the solution by adsorption onto a paper surface, and drying at 37° C. onto the surface. The application process may involve dipping, brushing, or spraying. Upon evaporation of the water, the ingredients will be covalently attached to the surface.

Test strip 10 is also composed of materials having sufficient rigidity and material properties such that it operates at an operating temperature range of 2 C to 80 C; can be stored at a temperature range of −20 C to 70 C; can withstand mechanical vibration of 50 Hz for 30 minutes; can withstand mechanical shock of 1 g; shall withstand thermal shock of −20 C to 80 C; and withstand handling shock of a 20 lb drop test from a height of 1 meter.

### Example

A base formulation of the solution to be applied to the strip was prepared using the components of Table 1. The components were added together as follows. Prepare the PPD solution by adding the potassium phosphate dibasic to water with stirring. Prepare the BSA solution by adding the bovine serum albumin to PPD at 37° C. with stirring. Prepare the reactant solution by adding the tetranitro blue, the nicotinamide adenine dinucleotide, nicotinamide adenine dinucleotide, the alcohol oxidase, the formaldehyde dehydrogenase, and the phenazine methosulfate to the BSA solution with stirring to form a yellow solution.

TABLE 1

| COMPONENT | WT. % |
|---|---|
| Water | 66.7869% |
| Bovine serum albumin [BSA] | 33.0000% |
| Tetranitro blue | 0.0800% |
| Nicotinamide adenine dinucleotide | 0.1000% |
| Alcohol oxidase from Pichia pastoris | 0.0010% |
| Formaldehyde dehydrogenase from Pseudomonas putida | 0.0320% |
| phenazine methosulfate | 0.0001% |

A sample of the formulation was applied to paper, and a fan was used to dry the paper while it was heated at 37° C. Subsequent rinsing of the paper with distilled water did not result in color transfer to the water. It can be seen that when the paper is stored dry, it has an excellent shelf life (i.e. stable form at least one year).

Whereas an embodiment of this invention has been described above for purposes of illustration, it will be evident to those skilled in the art that numerous variations of the details of the present invention may be made without departing from the invention as defined in the appended claims.

It is understood that the specific order of steps in the process disclosed is an illustration of an exemplary approach. Based on design preferences, it is understood that the specific order in the process may be rearranged. Some steps may be performed simultaneously. The accompanying method claims present elements of the various steps in a sample order and are not meant to be limited to the specific order. A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as an “aspect” may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such as “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as a “configuration” may refer to one or more configurations and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A test strip for colorimetrically indicating the presence of methanol in a solution it is in contact with, the test strip comprising:
a. a reagent to oxidize methanol to formaldehyde,
b. a reagent to oxidize formaldehyde to formic acid,
c. a reagent to be reduced in the formaldehyde oxidation,
d. a reagent to catalyze the reduction of a dye,
e. a dye that changes color when reduced.

2. The test strip in claim 1, wherein the reagent used to oxidize methanol to formaldehyde is present in an amount of 0.001% to 1.000%.

3. The test strip in claim 2, wherein the reagent used to oxidize methanol to formaldehyde is selected from one of the following: alcohol oxidase from Pichia pastoris, alcohol oxidase from Candida methanosorbosa, alcohol dehydrogenase from yeast, (2,2,6,6-tetramethylpiperidin-1-yl)oxyl (TEMPO), 2-azaadamantane-N-oxyl (AZADO), or 9-azabicyclo[3.3.1]nonane N-oxyl (ABNO).

4. The test strip in claim 1, wherein the reagent used to oxidize formaldehyde to formic acid is present in an amount of 0.01 to 1.00%.

5. The test strip in claim 4, wherein the reagent used to oxidize formaldehyde to formic acid is selected from: formaldehyde dehydrogenase from Pseudomonas putida, Methylococcus capsulatus, Pichia pastoris, Bacillus methanolicus, or Saccharomyces.

6. The test strip in claim 1, wherein the reagent to be reduced in the formaldehyde oxidation is present in an amount of 0.001% to 1.00%.

7. The test strip in claim 6, wherein the reagent to be reduced in the formaldehyde oxidation is nicotinamide adenine dinucleotide.

8. The test strip in claim 1, wherein the reagent to catalyze the reduction of a dye is present in an amount of 0.00001% to 1.00%.

9. The test strip in claim 8, wherein the reagent to catalyze the reduction of a dye is selected from: diaphorase from Clostridium kluyveri, Bacillus stearothermophilus, Bacillis megaterium, Escheria coli, Sus, Homo sapiens, or Rattus, or phenazine methosulfate.

10. The test strip in claim 1, wherein a dye that changes color when reduced is present in an amount of 0.001% to 1.00%.

11. The test strip in claim 10, wherein the dye that changes color when reduced is selected from: nitro blue, tetranitro blue, methylene blue, or 2,6-dichlorophenolindophenol.

12. The test strip in claim 1, wherein the colormetric indication occurs in less than or equal to 120 seconds when the presence of methanol is detected.

13. The test strip in claim 1, wherein the device shall not detect the presence of ethanol.

14. The test strip in claim 1, having an operating temperature range of 2 C to 80 C.

15. The test strip in claim 1, having a storage temperature range of −20 C to 70 C.

16. The test strip in claim 1, wherein it can withstand mechanical vibration of 50 Hz for 30 minutes.

17. The test strip in claim 1, wherein it can withstand mechanical shock of 1 g.

18. The test strip in claim 1, wherein the device shall withstand thermal shock of −20 C to 80 C.

19. The test strip in claim 1, wherein the device shall withstand handling shock of a 20 lb drop test from a height of 1 meter.
